(19) United States
(12) Patent Application Publication
(10) Pub. No.: US 2019/0152877 A1
(43) Pub. Date: May 23, 2019

(54) SELECTIVE PARTIAL HYDROGENATION OF TERPENES USING AN IRIDIUM-BASED CATALYST

(71) Applicants: CENTRE NATIONAL DE LA RECHERCHE SCIENTIFIQUE (CNRS), Paris (FR); TOTAL RAFFINAGE CHIMIE, Courbevoie (FR)

(72) Inventors: Chloé THIEULEUX, Villeurbanne (FR); Emmanuel LACÔTE, Paris (FR); Delphine CROZET, Villeurbanne (FR)

(73) Assignees: CENTRE NATIONAL DE LA RECHERCHE SCIENTIFIQUE (CNRS), Paris (FR); TOTAL RAFFINAGE CHIMIE, Courbevoie (FR)

(21) Appl. No.: 15/571,345

(22) PCT Filed: May 6, 2016
(86) PCT No.: PCT/EP2016/060152

§ 371 Date: Nov. 2, 2017

(30) Foreign Application Priority Data
May 7, 2015 (EP) 15305703.9

(51) Int. Cl. C07C 5/05; C07C 11/02; B01J 31/16; B01J 31/22

(52) U.S. Cl. CPC C07C 5/05; C07C 11/02; B01J 31/1633; B01J 35/1028; B01J 2231/645; B01J 2531/827; B01J 31/2273

(57) ABSTRACT

A process for selective partial hydrogenation of conjugated diene compounds includes at least one, preferably terminal, diene function and at least one additional carbon-carbon double bond, the process including reacting the conjugated diene compounds with hydrogen in the presence of an iridium-NHC based catalyst. The disclosure also relates to a reaction mixture that can be obtained at the end of the process. The disclosure also relates to the use of the reaction mixture.

### FIELD OF THE INVENTION

The invention relates to a process for the selective partial hydrogenation of conjugated diene compounds having at least one conjugated diene function and at least one additional carbon-carbon double bond in order to produce partially hydrogenated compounds, in particular alpha-olefins.

The invention also relates to reaction mixtures that can be obtained at the end of the process of the invention. The invention also relates to the use of the reaction mixtures of the invention.

### BACKGROUND OF THE INVENTION

Olefins can be used as raw materials in different processes. Depending on the processes, different olefins may be used. For example, alpha-olefins can be easily functionalized and used in different industrial processes. Mono-olefins, di-olefins or tri-olefins may be useful as raw materials in different processes, in particular in different kinds of reactions.

As a result of the increasing scarcity of fossil resources and of ever-increasing environmental concerns, the use of molecules derived from biomass is increasingly sought to replace molecules of fossil origin. Up to now, molecules of fossil origin are widely used in the production of fuels or technical fluids, such as lubricants, drilling fluids or solvents.

There is now a continuous trend to manufacture fuels and technical fluids thanks to molecules derived from biomass, such as terpenes.

There is a need for renewable olefinic feedstocks that are not derived from fossil fuels. Furthermore, there is a need for alternate olefinic feedstocks, in particular olefinic feedstocks that do not include detectable amounts of sulfur or aromatic compounds. Additionally, there is a need for methylated olefinic feedstocks, in particular olefinic feedstocks in which the methylation position is controlled.

In the polymerization technologies, there is also a need to find alternatives, potentially bio-sourced, for replacement of polyisoprenes as well as for the chain transfer agent isoamylene.

Partial hydrogenation of olefinic feedstocks, in particular renewable olefinic feedstocks, allows manufacturing different olefins, such as mono-olefins, di-olefins or tri-olefins, which may subsequently be used as raw materials in different industrial processes. The partial hydrogenation should be selective in order to control the obtained composition and facilitate the separation of the partially hydrogenated compounds that may be made after the partial hydrogenation reaction.

There is thus a need for the selective hydrogenation of terpenes.

The selective hydrogenation of myrcene has been reported with complexes of ruthenium, chromium, iridium and rhodium. One neutral iridium complex [IrCl(CO)(PPh₃)₂] is described as active for the hydrogenation of myrcene (Journal of Molecular Catalysis A: Chemical 239 (2005) 10-14). The Article of MG Speziali et al in Journal of Molecular Catalysis A: Chemical 239 (2005) 10-14 discloses a reaction mixture comprising four different mono-hydrogenated compounds, said mono-hydrogenated compounds are not characterized in that one mono-hydrogenated compound represents at least 50% by weight of the weight of all the mono-hydrogenated compounds. The selectivity discloses in said document is not as good as the selectivity obtained by the process of the present invention.

Cationic Iridium complexes have been reported for the selective hydrogenation of carbon-carbon multiple bonds of functionalized alkenes and alkynes or the hydrogenation of mono-unsaturated alkenes (Chem. Commun., 2011, 47, 11653-11655) and catalytic isotope exchange (Chem. Commun., 2008, 1115-1117).

Document WO 2012/141783 describes the manufacture of partially hydrogenated molecules from conjugated alkenes. However this document allows obtaining a mixture of mono-, di- or tri-hydrogenated molecules and among them all the isomers for each molecular mass are formed. For example, for the farnesene, the process disclosed in this document leads to a reaction mixture comprising several isomers of molecular mass 206, several isomers of molecular mass 208 and several isomers of molecular mass 210.

There is thus a need for a process leading to partially hydrogenated products with an improved selectivity.

Selective hydrogenation of poly-unsaturated alkenes such as farnesene can be highly difficult using either classical heterogeneous or homogeneous catalysts, since the hydrogenation is very active or only allows obtaining a mixture of products with competitive isomerization reaction. Among selective catalyst complexes, most often used complexes are homogenous ones. However, deactivation processes can be reported, such as the formation of polynuclear hydride species for iridium catalysis. One solution is the isolation of transition metal complexes on silica surface.

Document WO 2009/092814 describes organometallic materials that can be used as heterogeneous catalyst. This document does not disclose the selective partial hydrogenation of conjugated diene compounds.

### SUMMARY OF THE INVENTION

A first object of the present invention is a process for the partial hydrogenation of conjugated diene compounds comprising at least one conjugated diene function and at least one additional carbon-carbon double bond, said process comprising reacting the conjugated diene compounds with hydrogen in the presence of a Iridium-NHC based catalyst, to produce a reaction mixture comprising partially hydrogenated compounds.

According to a preferred embodiment, the at least one conjugated diene function of the conjugated diene compounds is a terminal conjugated diene function.

According to a preferred embodiment, a portion of the partially hydrogenated compounds results from the mono-hydrogenation of one carbon-carbon double bond of the conjugated diene function.

According to a preferred embodiment, a portion of the partially hydrogenated compounds results from the di-hydrogenation of the two carbon-carbon double bonds of the conjugated diene function.

Preferably, the conjugated diene compounds comprising at least one conjugated diene function and at least one additional carbon-carbon double bond are selected from terpenes, more preferably from myrcene and farnesene.

According to an embodiment of the invention, the hydrogenation is performed at a temperature ranging from 10° C. to 120° C., preferably from 20° C. to 110° C.

According to an embodiment of the invention, the hydrogenation is performed at a pressure ranging from 2 bars to 12 bars, preferably from 3 bars to 10 bars.

According to an embodiment of the invention, the reaction mixture comprises mono-hydrogenated compounds wherein a mono-hydrogenated compound resulting from the hydrogenation of one carbon-carbon double bond of the conjugated diene function represents at least 50% by weight of the total weight of the mono-hydrogenated compounds.

According to an embodiment of the invention, the conjugated diene compound is a compound of formula (g):

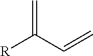

wherein, R is a hydrocarbyl radical having 1 to 40 carbon atoms and comprising at least one carbon-carbon double bond, optionally comprising one or more heteroatoms, such as nitrogen, oxygen or sulphur,

Preferably, the partially hydrogenated compounds are characterized in that:
the compound of formula (g1) or (g5) represents at least 50% by weight, preferably at least 60% by weight, more preferably at least 70% by weight, even more preferably at least 80% by weight, of the total weight of the mono-hydrogenated compounds, wherein

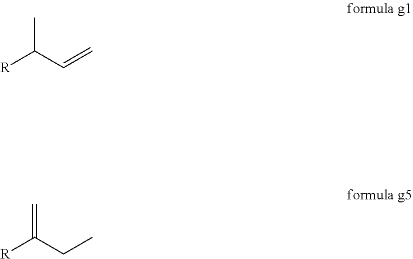

the compound of formula (g2) represents at least 50% by weight, preferably at least 60% by weight, more preferably at least 70% by weight, even more preferably at least 80% by weight, of the total weight of the di-hydrogenated compounds, wherein

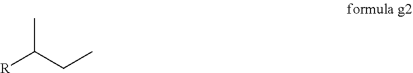

In formulas (g1), (g5) and (g2), R represents the same group as in formula (g).

According to an embodiment of the invention, the iridium-NHC based catalyst is a homogeneous catalyst.

Preferably, the homogeneous catalyst is a complex of formula (I) or of formula (II):

[Ir(L1)(L2)(NHC)(L3)]X (I)

[Ir(L′1)(L′2)(NHC)(L′3)] (II)

wherein

L1, L2 and L3 are independently to each other a ligand,

L′1, L′2 and L′3 are independently to each other a ligand,

X is a non-coordinated counter-anion.

According to an embodiment of the invention, the iridium-NHC based catalyst is a heterogeneous catalyst.

Preferably, the heterogeneous catalyst is a silica-supported iridium-NHC based catalyst of formula (III):

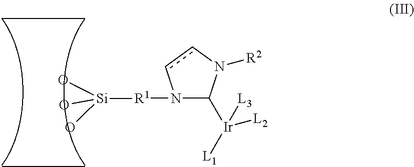

wherein:

L₁, L₂ and L₃ are independently to each other a ligand,

R¹ represent an alkylene or an arylene group optionally substituted, and

R² represents an alkyl or an aryl group optionally substituted.

Preferably, when the catalyst is of formula (I), the reaction mixture is such that the compound of formula (g1) represents at least 50% by weight, preferably at least 60% by weight, more preferably at least 70% by weight, of the total weight of the mono-hydrogenated compounds.

A second object of the invention is a reaction mixture obtainable by the process of the invention, said reaction mixture comprises:
from 20 to 80% by weight of compound(s) A resulting from the mono-hydrogenation of compounds of formula (g),
from 20 to 80% by weight of compound(s) B resulting from the di-hydrogenation of compounds of formula (g),

based on the total weight of the partially hydrogenated compounds,

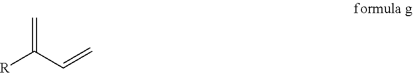

wherein
R is a hydrocarbyl radical having 1 to 40 carbon atoms and comprising at least one carbon-carbon double bond, optionally comprising one or more heteroatoms, such as nitrogen, oxygen or sulphur,
   at least 50% by weight based on the total weight of the compounds A is represented by a compound of formula (g1) or a compound of formula (g5)

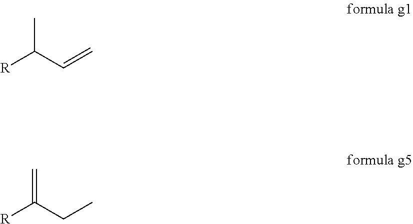

at least 70% by weight based on the total weight of the compounds B is represented by a compound of formula (g2)

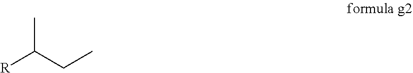

in formulas (g1), (g2) and (g5), R has the same meaning as in formula (g).

According to an embodiment of the invention, the R group of the compounds of formula (g) is a hydrocarbyl radical having 1 to 40 carbon atoms and comprising at least two carbon-carbon double bonds, optionally comprising one or more heteroatoms, such as nitrogen, oxygen or sulphur,

and said reaction mixture comprises:
from 5 to 70% by weight, preferably from 20 to 70% by weight, more preferably from 30 to 60% by weight, of compound(s) A resulting from the mono-hydrogenation of compounds of formula (g),
from 20 to 80%, preferably from 30 to 70% by weight, more preferably from 40 to 60% by weight, by weight of compound(s) B resulting from the di-hydrogenation of compounds of formula (g), and
from 10 to 70% by weight, preferably from 20 to 60% by weight, more preferably from 30 to 50% by weight, of compound(s) C resulting from the tri-hydrogenation of compounds of formula (g),
based on the total weight of the partially hydrogenated compounds.

Preferably, the compound of formula (g) is farnesenes, said reaction mixture comprises:
from 5 to 70% by weight, preferably from 20 to 70% by weight, more preferably from 30 to 60% by weight, of compound(s) A resulting from the mono-hydrogenation of farnesene,
from 20 to 80%, preferably from 30 to 70% by weight, more preferably from 40 to 60% by weight, by weight of compound(s) b resulting from the di-hydrogenation of farnesene, and
from 10 to 70% by weight, preferably from 20 to 60% by weight, more preferably from 30 to 50% by weight, of compound(s) C resulting from the tri-hydrogenation of farnesene,

based on the total weight of the partially hydrogenated farnesene,
wherein at least 50% by weight based on the total weight of the mono-hydrogenated farnesene is represented by a compound of formula f1 or f5:

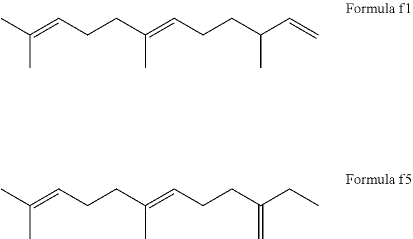

and wherein at least 70% by weight based on the total weight of the di-hydrogenated farnesene is represented by a compound of formula f2:

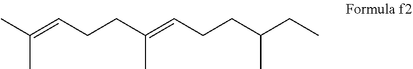

A further object of the present invention is the use of the reaction mixture of the invention or a derivative thereof, in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as EDM (Electronic Discharge Machining) fluids, rust preventive in industrial lubricants, as extender oils, as drilling fluids, as industrial solvents, as viscosity depressants in plasticized polyvinyl chloride formulations, as crop protection fluids.

The process of the invention is simple and allows providing desired products with a high selectivity.

Further features and advantages of the invention will appear from the following description of embodiments of the invention, given as non-limiting examples.

### DETAILED DESCRIPTION OF THE INVENTION

### Process for the Partial Hydrogenation

The present invention is directed to a process for the partial hydrogenation of conjugated diene compounds comprising at least one conjugated diene function and at least one additional carbon-carbon double bond, said process comprising reacting the conjugated diene compounds with hydrogen in the presence of a Iridium-NHC based catalyst, to produce a reaction mixture comprising partially hydrogenated compounds, preferably a portion of said partially hydrogenated compounds resulting from the mono-hydrogenation of one carbon-carbon double bond of the terminal conjugated diene function.

Conjugated Diene Compounds

The conjugated diene compounds that are hydrogenated according to the process of the invention comprise at least one conjugated diene function and at least one additional carbon carbon-double bond.

The at least one conjugated diene function of the conjugated diene compound may be either terminal conjugated diene function or not-terminal conjugated diene function.

The conjugated diene compound may be represented by the following formula (g):

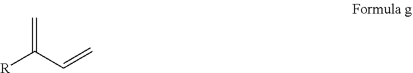

wherein R is a hydrocarbyl radical having 1 to 40 carbon atoms and comprising at least one carbon-carbon double bond, optionally comprising one or more heteroatoms, such as nitrogen, oxygen or sulphur.

Preferably, R is a hydrocarbyl radical having from 5 to 20 carbon atoms and comprising at least one carbon-carbon double bond, optionally comprising one or more heteroatoms, such as nitrogen, oxygen or sulphur.

According to a specific embodiment, R consists in carbon and hydrogen atoms.

The conjugated diene compounds may comprise only one kind of conjugated diene compound or a mixture of different conjugated diene compounds. Preferably, the conjugated diene compounds, as starting product of the process, comprise only one kind of conjugated diene compound.

The conjugated diene compounds, as starting mixture of the process, generally consist essentially of conjugated diene compounds. Very few impurities may be present in the conjugated diene compounds. Preferably, conjugated diene compounds comprise at least 95% by weight of conjugated diene compounds, more preferably at least 97% by weight, even more preferably at least 99% by weight, based on the total weight of conjugated diene compounds.

According to an embodiment, the conjugated diene compounds are chosen from terpenes, preferably from terpenes having from 10 to 40 carbon atoms.

Terpenes are molecules of natural origin, produced by numerous plants, in particular conifers.

By definition, terpenes (also known as isoprenoids) are a class of hydrocarbons bearing as the base unit an isoprene moiety (i.e. 2-methyl-buta-1,3-diene). Isoprene [CH₂═C(CH₃)CH═CH₂] is represented below:

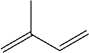

Terpenes may be classified according to the number n (integer) of isoprene units of which it is composed, for example:

n=2: monoterpenes (C₁₀), such as myrcene or pinene (alpha or beta), are the most common;

n=3: sesquiterpenes (C₁₅), such as farnesene;

n=4: diterpenes (C₂₀);

n=5: sesterpenes (C₂₅);

n=6: triterpenes (C₃₀), such as squalene;

n=7: tetraterpenes (C₄₀), such as carotene (C₄₀H₆₄), which is an important pigment of plant photosynthesis.

Many isomers exist in each of the families.

The carbon backbone of terpenes may consist of isoprene units arranged end to end to form linear molecules. The arrangement of the isoprene units may be different to form a branched or cyclic backbone.

Preferably, terpenes are chosen from myrcene and farnesenes, preferably from farnesenes, in particular from beta-farnesene.

Beta-farnesene refers to a compound having the following formula (f):

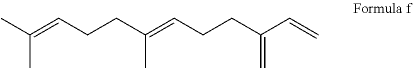

Myrcene refers to a compound having the following formula (m):

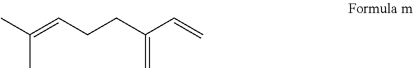

As another example of the conjugated diene compound responding to formula (g), mention may be made of farnesene epoxide:

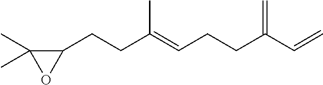 [SMILES: C=CC(=C)CC/C=C(\C)CCC1OC1(C)C]

Catalyst Used in the Process

The catalyst used in the present invention in order to perform the selective hydrogenation reaction is chosen from Iridium-NHC based catalysts.

According to the present invention, NHC refers to a N-heterocyclic carbene and corresponds to a 1,3-di-substituted-imidazol-2-ylidene (R¹R²Im).

In particular, NHC responds to the following formula:

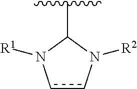

wherein,

the free valence (symbolized by [symbol] ) of the NHC is linked to the metal atom of the catalyst,

the carbon-carbon bond in the NHC cycle can be either a carbon-carbon double bond or a carbon-carbon simple bond, preferably the carbon-carbon bond in the NHC cycle is a carbon-carbon double bond,

R¹ and R² represent independently to each other, an alkyl or an aryl group optionally substituted.

Preferably, R¹ and R² are, independently to each other, selected from the group consisting of C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether. R¹ and/or R² may also represent C₅₋₂₀-aryl substituted with one or more moieties selected from oxazoline, substituted oxazoline, pyrazoline or substituted pyrazoline.

According to the present invention, the catalyst may be supported or not supported. Indeed, the process according to the present invention may be performed by homogeneous catalysis (i.e. the catalyst is soluble in the reaction medium) or heterogeneous catalysis (i.e. the catalyst is not soluble in the reaction medium).

When supported, the support may be chosen from silica.

The iridium-NHC based catalyst may be in the form of a cationic or a neutral complex.

Preferably, the iridium-NHC based catalyst is in the form of a cationic complex.

According to an embodiment of the invention, the iridium-NHC based catalyst responds to the formula (I):

[Ir(L1)(L2)(NHC)(L3)]X (I)

wherein L1, L2 and L3 represent independently a ligand and X represents a non-coordinating counter-anion. According to this formula, the iridium catalyst is a cationic complex.

L1, L2, L3 may be, independently to each other, chosen from 1,5-cyclooctadiene, halogen, phosphane or solvent molecules.

Indeed, it is possible that the solvent molecule optionally used in the hydrogenation process coordinates with the metal. For example, if methanol is used as a solvent, the methanol molecule through its oxygen atom may play the role of a ligand.

L1 and L2 may correspond to the 1,5-cyclooctadiene (COD) or may be chosen from halogen, such as chlorine or a iodine atom. L3 may be chosen from phosphine compounds, such as triphenylphosphine (PPh₃), tribenzylphosphane (PBn₃), dimethylphenylphosphine (PMe₂Ph).

X may be a hexafluorophosphate (PF₆⁻), tetrafluoroborate (BF₄⁻), [B[3,5-(CF₃)₂C₆H₃]₄]⁻ anion (commonly abbreviated as [BArF₄]⁻) or perchlorate ion (ClO₄⁻).

Preferably, the cationic iridium complex responds to the formula (Ibis):

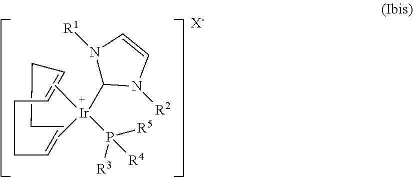

wherein

R¹ and R² represents independently to each other an alkyl or an aryl group optionally substituted, preferably R¹ and R² are selected from the group consisting of C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether;

X⁻ represents a non-coordinated counter-anion, preferably X⁻ is chosen from hexafluorophosphate (PF₆⁻), tetrafluoroborate (BF₄⁻), [B[3,5-(CF₃)₂C₆H₃]₄]⁻ anion (commonly abbreviated as [BArF₄]⁻) or perchlorate ion (ClO₄⁻).

The cationic iridium complex of formula (I) or (Ibis) may be obtained according to a method known by the skilled person, such as the method described in Chem. Commun., 2011, 47, 11653-11655. They are commercially available, for example by Strem Chemicals Company.

R³, R⁴ and R⁵ are independently to each other chosen from alkyl or aryl groups optionally substituted, preferably from an alkyl having from 2 to 12 carbon atoms or an aryl having from 6 to 12 carbon atoms.

According to another embodiment of the invention, the iridium-NHC based catalyst responds to the formula (II):

[Ir(L′1)(L′2)(NHC)(L′3)] (II)

wherein L′1, L′2 and L′3 are independently to each other a ligand. According to this formula, the iridium catalyst is a neutral complex.

L′1, L′2, L′3 may be, independently to each other, chosen from 1,5-cyclooctadiene, halogen, phosphane or solvent molecules.

Indeed, it is possible that the solvent molecule optionally used in the hydrogenation process coordinates with the metal. For example, if methanol is used as a solvent, the methanol molecule through its oxygen atom may play the role of a ligand.

L′1 and L′2 may correspond to the 1,5-cyclooctadiene (COD). L′3 may be a halogen atom, such as a chlorine atom or a iodine atom.

The iridium-NHC based catalyst of formula (II) may be obtained by a method known for the skilled person, such as a method described in J. P. Collman, C. T. Sears Jr., M. Kubota, Inorg. Synth. 28 (1990) 92.

According to another embodiment of the invention, the iridium-NHC based catalyst is a silica-supported catalyst and responds to the formula (III):

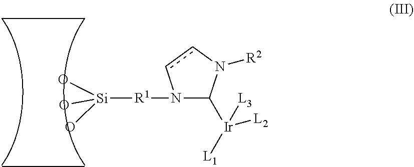

wherein:

L₁, L₂ and L₃ are independently to each other a ligand,

R¹ is a divalent linker, for example R¹ is chosen from alkylene, arylene group, substituted or not,

and R² represents an alkyl or an aryl group optionally substituted.

Preferably, R¹ is selected from the group consisting of C₁₋₂₀-alkylene, C₅₋₂₀-arylene, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether.

Preferably, R² is selected from C₁₋₂₀-alkyl, C₅₋₂₀-aryl, which can be optionally substituted with one or more moieties selected from the group consisting of C₁₋₁₀-alkoxy, phosphine, sulfonated phosphine, phosphate, phosphinite, arsine, ether, amine, amide, imine, sulfoxide, carboxyl, nitrosyl, pyridine, substituted pyridine, imidazole, substituted imidazole, pyrazine, substituted pyrazine and thioether, oxazoline, substituted oxazoline, pyrazoline, substituted pyrazoline.

According to an embodiment, R² may interact with the metal atom, through for example a coordination bond. In particular, if the ligand is weakly coordinated, the ligand may be replaced by the R² radical.

As it is well known for the skilled person, the support illustrated in the above formula (III) is a schematic illustration, such that a support comprises one or several metal atoms.

In the above-formula of the supported catalyst, the iridium center may be either neutral or cationic. In the case where the iridium is cationic, the catalyst contains a non-coordinated counter-anion (see the X⁻ group defined above).

According to an embodiment, in the above-formula (III), L₁, L₂ and L₃ are selected from halogen, such as chlorine, 1,5-cyclo-octadiene (COD), phosphane ligand, solvent molecule or surface interaction. Indeed, the surface of the support (for example the silica) or the solvent may act as a ligand. In particular, the interaction with the surface may be made thanks to the oxygen atoms.

The supported iridium-NHC based catalysts of formula (III) were found to be extremely active in the reaction of hydrogenation. Surprisingly, the catalytic activity is better than the catalytic activity of similar homogeneous complexes.

Preferably, in the above-formula (III) of the supported Ir—NHC based catalyst, the carbon-carbon bond in the NHC cycle is a carbon-carbon double bond, which corresponds to the catalyst of formula (IlIbis) as defined below:

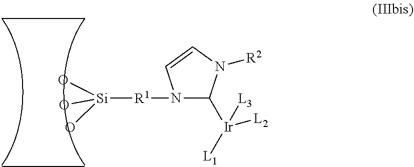

The silica-supported catalyst of formula (III) may be obtained according to a process described hereinafter for the silica-supported catalyst of formula (IlIbis).

The catalyst of formula (IlIbis) above may be obtained according to a method described in document WO 2009/092814.

For example, the catalyst may be obtained according to the following method:

In a first step, a chloropropyltriethoxysilane may react with a sodium iodide in order to form a iodipropyltriethoxysilane:

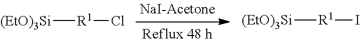

In a second step, there is a step of hydrolysis-polycondensation with for example a Pluronic® 123 as structure-directing agent:

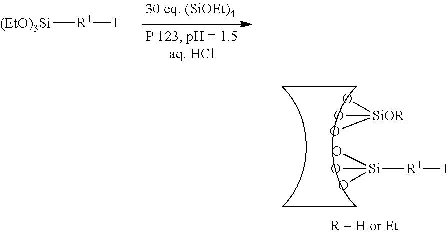

The amount of SiOEt₄ may range from 20 to 200 molar equivalents with respect to (EtO)₃SiR¹I.

The above scheme is only illustrative in order to represent a pore of the support. Indeed, another manner of representation may be: IR¹SiO_{1.5}/30SiO₂.

Then, there is a step of functionalization with a derived of imidazole:

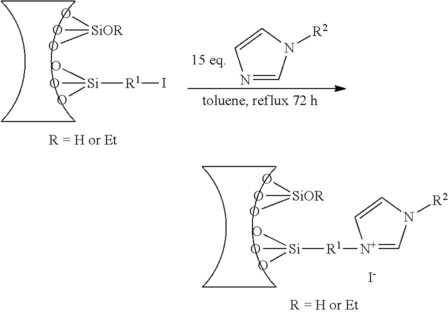

Optionally, if the R group is not a hydrogen group, there is a step of hydrolysis:

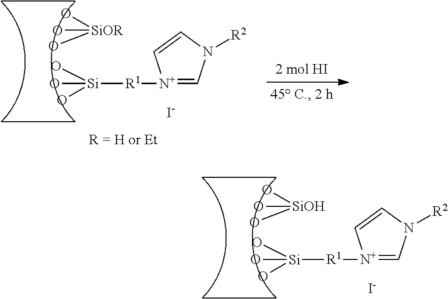

Then, a passivation step may be performed to transform the surface silanols groups into trimethoxysiloxane groups. This step is optional:

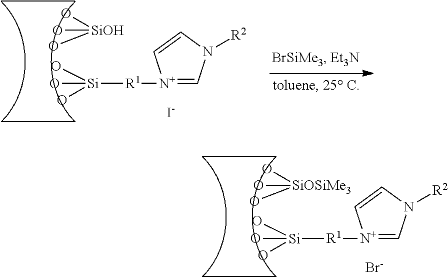

In a further step, the imidazolium-containing material is treated with AgOC(CF₃)₃ to give a silver-NHC supported complex:

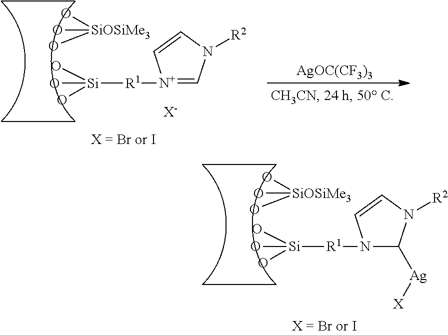

X⁻, depending on the presence or not of the optional passivation step detailed above, may represent either Br⁻ or I⁻.

Finally, the supported iridium-NHC complex according to the invention may then be obtained from the silver-NHC complex by the following reaction conditions:

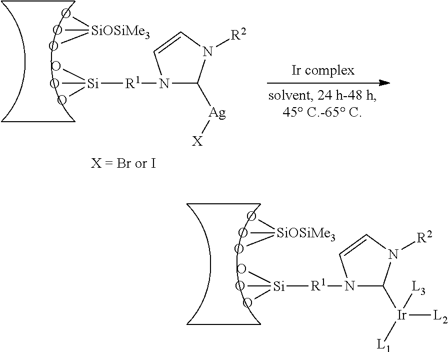

Among iridium complexes, mention may be made of: [Ir(L1)(L2)(L3)], where L₁, L_{2,} L₃ represent ligands, preferably selected from halogen, such as chlorine, 1,5-cyclo-octadiene (COD), phosphane ligand, solvent molecule or surface interaction.

As an example of iridium complex, mention may be made of [Ir(COD)(Cl₂)], COD being a bidentate cyclo-octadiene ligand. Said iridium complex allows obtaining a supported neutral iridium complex without phosphine. In the final supported neutral complex, L₁, L₂ and L₃ may be Cl or 1,5-cyclo-octadiene or surface interaction or solvent molecules.

The supported iridium complex of formula (III) or (IlIbis) may be a supported cationic iridium complex.

The supported cationic iridium complex may be represented by the following formula (IV):

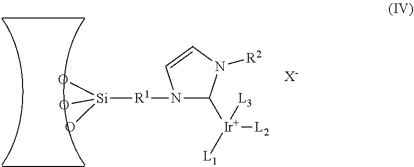

In the above formula (IV), R¹ and R² have the same meaning as in formula (III) and (IlIbis); L₁, L₂ and L₃ are independently to each other a ligand, preferably selected from 1,5-cyclo-octadiene (COD), phosphane ligand, solvent molecules or surface interaction.

The supported cationic iridum complex may be obtained in a medium containing acetone, at 25° C. during 3 h by using AgBF₄ and a phosphane ligand, according to the following scheme given as a specific example:

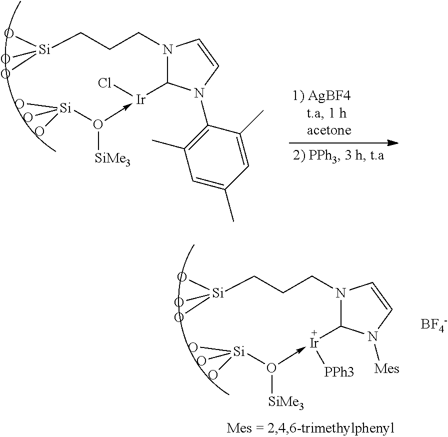

In the above scheme, the surface plays the role of ligand.

According to an embodiment, the supported catalyst of formula (III) or (IIIbis) or (IV) that can be used in the process of the invention has the following characteristics:

The material may exhibit an N2 adsorption-desorption isotherm at 77 K of type IV, from 300 to 1200 m²/g, for example of 1146 m²/g, which is characteristic of mesoporous materials, with a large BET specific surface area.

The material may have a pore volume (Vp) ranging from 0.5 to 1.5 cm3/g, for example of around 1.4 cm³/g.

The material may also exhibit a mean pore diameter (D_{pBJII}) ranging from 3 to 25 nm, for example of 5.7 nm.

The TEM and powder XRD measurements are consistent with a material having a long-range structuration of the pore network with a 2D hexagonal array. ¹³C solid state NMR spectroscopy confirms the presence of the functional groups. The ²⁹Si NMR spectrums show the characteristic signals corresponding to the organic units bounded to the matrix via three Si—O bonds and to the degree of condensation of the material.

The Iridium-NHC containing materials are classically described by X-ray diffraction, elemental analysis, N2 adsorption/desorption, TEM and ¹H, ¹³C, and ²⁹Si solid-state NMR spectroscopy.

Hydrogenation Process

The process of the present invention comprises a step of contacting the conjugated diene compounds with hydrogen in the presence of a specific catalyst, said conjugated diene compounds comprise at least one terminal conjugated diene function and at least one additional carbon-carbon double bond.

Preferably, the hydrogenation process is a one-step process, in particular said one-step process consists in the following: mixture of reactants, hydrogenation reaction and recovery of the reaction products.

The process of the present invention leads to a reaction mixture comprising partially hydrogenated compounds. In particular, a portion of said partially hydrogenated compounds being mono-hydrogenated compounds wherein one carbon-carbon double bond of the conjugated diene function has been hydrogenated.

By mono-hydrogenated compound, it is to be understood a compound wherein only one carbon-carbon double bond has been hydrogenated.

By di-hydrogenated compound, it is to be understood a compound wherein two carbon-carbon double bonds have been hydrogenated.

By tri-hydrogenated compound, it is to be understood a compound wherein three carbon-carbon double bonds have been hydrogenated.

By the expression “the mono-hydrogenated compounds mainly comprise a specific compound”, it is to be understood that said specific compound represents at least 50% by weight, preferably at least 60% by weight, more preferably at least 70% by weight, even more preferably at least 80% by weight of the total weight of the mono-hydrogenated compounds.

By “reaction mixture”, it is to be understood the olefinic mixture that is obtained at the end of the hydrogenation process. The reaction mixture may comprise the partially hydrogenated compounds, conjugated diene compounds that have not reacted, fully hydrogenated compounds, by-products (i.e. products obtained by side reactions different from a hydrogenation reaction) and an optional solvent.

By the expression “the reaction mixture mainly comprises compound(s)”, it is to be understood that said compound(s) represents at least 50% by weight, preferably at least 60% by weight, more preferably at least 70% by weight, even more preferably at least 80% by weight, of the total weight of the reaction mixture.

By “partially hydrogenated compounds”, it is to be understood unsaturated hydrogenated compounds, i.e. hydrogenated compounds comprising at least one carbon-carbon double bond.

By “the Process is Selective”, it is to be Understood that the Process Leads to:

i) a reaction mixture comprising partially hydrogenated compounds comprising mono-hydrogenated compounds characterized in that said mono-hydrogenated compounds comprise in majority a specific isomer among different existing isomers resulting from a mono-hydrogenation of the conjugated diene compounds comprising one terminal diene function and at least one additional carbon-carbon double bond., or

ii) a reaction mixture comprising partially hydrogenated compounds comprising in majority di-hydrogenated compound(s).

By the term ‘in majority”, it is to be understood in a proportion of at least 50% by weight, preferably at least 60% by weight, more preferably at least 70% by weight, even more preferably at least 80% by weight.

According to an embodiment of the invention, the partially hydrogenated compounds comprise one or more mono-hydrogenated compounds characterized in that a specific mono-hydrogenated compound represents at least 50% by weight of the total weight of the mono-hydrogenated compounds. Preferably, said specific mono-hydrogenated compound represents at least 60% by weight, more preferably at least 70% by weight, even more preferably at least 80% by weight, of the total weight of the mono-hydrogenated compounds.

Preferably, the mono-hydrogenated compound representing at least 50% by weight of the mono-hydrogenated compounds obtained in the reaction mixture is a mono-hydrogenated compound resulting from the hydrogenation of one carbon-carbon double bond of the conjugated diene function, preferably from the hydrogenation of the carbon-carbon double bond of the conjugated diene function in terminal position.

According to an embodiment of the invention, the partially hydrogenated compounds comprise mono-hydrogenated compounds and di-hydrogenated compounds.

Preferably, the reaction mixture comprises:
from 10 to 80% by weight, preferably from 20 to 70% by weight, more preferably from 30 to 60% by weight, of mono-hydrogenated compounds,
from 20 to 90%, preferably from 30 to 80% by weight, more preferably from 40 to 70% by weight, by weight of di-hydrogenated compounds, and

based on the total weight of the partially hydrogenated compounds.

According to an embodiment, the starting conjugated diene compounds comprise a terminal conjugated diene function and at least two carbon-carbon double bonds.

According to this embodiment, the partially hydrogenated compounds comprise mono-hydrogenated compounds, di-hydrogenated compounds and tri-hydrogenated compounds.

Preferably, the reaction mixture comprises:
from 10 to 70% by weight, preferably from 20 to 60% by weight, more preferably from 30 to 50% by weight, of mono-hydrogenated compounds,
from 20 to 80%, preferably from 30 to 70% by weight, more preferably from 40 to 60% by weight, by weight of di-hydrogenated compounds, and
from 10 to 70% by weight, preferably from 20 to 60% by weight, more preferably from 30 to 50% by weight, of tri-hydrogenated compounds,

based on the total weight of the partially hydrogenated compounds.

Among di-hydrogenated compounds, preferably, the mainly obtained di-hydrogenated compound is a specific di-hydrogenated compound wherein both conjugated carbon-carbon double bonds have been hydrogenated. In particular, according to this embodiment of the invention, the reaction mixture is such that said specific di-hydrogenated compound represents at least 50% by weight, preferably at least 60% by weight, more preferably at least 70% by weight, even more preferably at least 80% by weight, ideally at least 90% by weight, of the total weight of the di-hydrogenated compounds.

The process of hydrogenation of the invention is very selective, in particular thanks to the Iridium-NHC catalyst, it is possible to mainly obtain only one isomer among the different existing isomers resulting from the mono-hydrogenation of the conjugated diene compounds. It is also possible to obtain a specific di-hydrogenated compound among the different existing isomers resulting from the di-hydrogenation of the conjugated diene compounds, thanks to the iridium-NHC catalyst.

Thanks to the process of the invention, the reaction mixture may comprise at least 50% by weight, preferably at least 60% by weight, of a specific di-hydrogenated compound, based on the total weight of the partially hydrogenated compounds, said specific di-hydrogenated compound being the starting conjugated diene compound wherein the terminal conjugated diene function has been totally hydrogenated.

According to an embodiment of the invention, when the starting conjugated diene compound is a compound of formula (g) as detailed above, the reaction mixture obtained at the end of the process is such that the compound of formula (g1) or (g5) represents at least 50% by weight, preferably at least 60% by weight, more preferably at least 70% by weight, of the total weight of the mono-hydrogenated compounds, wherein

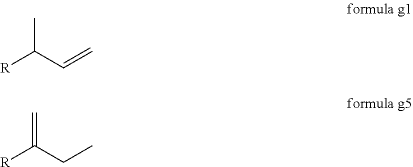

In formulas (g1) and (g5), R represents the same group as in formula (g).

In particular, when the iridium-NHC based catalyst is in the form of a cationic complex, the reaction mixture obtained at the end of the process of the invention comprises mono-hydrogenated compounds, said mono-hydrogenated compounds mainly comprising a compound resulting from the (mono-) hydrogenation of the carbon-carbon double bond in terminal position of the conjugated diene function.

Thanks to an iridium-NHC based catalyst in the form of a cationic complex, when the conjugated diene compound is of formula (g), the reaction mixture obtained at the end of the process is such that the compound of formula (g1) represents at least 50% by weight, preferably at least 60% by weight, more preferably at least 70% by weight, even more preferably at least 80% by weight, based on the total weight of the mono-hydrogenated compounds.

Thanks to an iridium-NHC based catalyst in the form of a neutral complex, when the conjugated diene compound is a compound of formula (g), the reaction mixture obtained at the end of the process is such that a compound of formula (g5) represents at least 50% by weight, preferably at least 60% by weight, more preferably at least 70% by weight, even more preferably at least 80% by weight, based on the total weight of the mono-hydrogenated compounds.

According to an embodiment of the invention, partially hydrogenated compounds comprise mono-hydrogenated compounds and di-hydrogenated compounds. According to this embodiment, if the conjugated diene compound is a compound of formula (g), the partially hydrogenated compounds comprise a compound of formula (g2) as the mainly obtained di-hydrogenated compound,

wherein

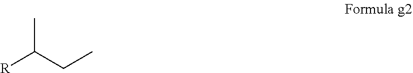

wherein R has the same meaning as in formula (g).

In particular, according to this embodiment of the invention, the reaction mixture is such that the compound of formula (g2) represents at least 50% by weight, preferably at least 60% by weight, more preferably at least 70% by weight, even more preferably at least 80% by weight, ideally at least 90% by weight, of the total weight of the di-hydrogenated compounds.

For example, when the starting conjugated diene compounds are farnesene, the process of the invention allows obtaining at least 50% by weight of a mono-hydrogenated compound (based on the total weight of the mono-hydrogenated compounds) which is either the compound of formula (f1) or the compound of formula (f5):

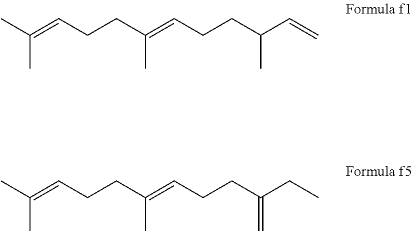

When the iridium-NHC based catalyst is in the form of a cationic complex, the compound of formula (f1) is mainly obtained, i.e. in a proportion such that the compound of formula (f1) represents at least 50% by weight of the total weight of the mono-hydrogenated compounds.

When the iridium-NHC based catalyst is in the form of a neutral complex, the compound of formula (f5) is mainly obtained, i.e. in a proportion such that the compound of formula (f5) represents at least 50% by weight of the total weight of the mono-hydrogenated compounds.

In the case wherein the conjugated diene compounds are farnesene, the process of the invention may also lead to other mono-hydrogenated compounds and/or to di-hydrogenated compounds and/or to tri-hydrogenated compounds.

Among the other mono-hydrogenated compounds derived from farnesene, mention may be made of the compounds of formula (f3) and of formula (f4):

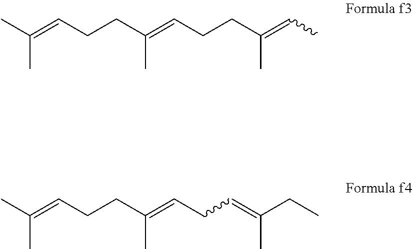

Among di-hydrogenated compounds derived from farnesene, mention may be made of the compound of formula (f2):

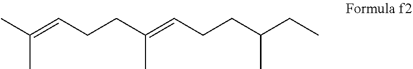

Among the tri-hydrogenated compounds derived from farnesene, mention may be made of the compound of formula (f7) and of formula (f8):

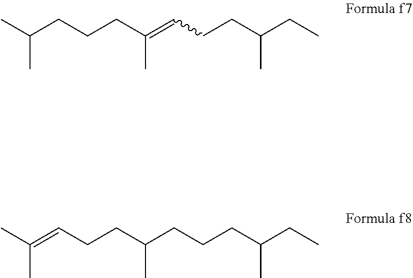

According to an embodiment, the reaction mixture comprises:
from 20 to 80% by weight, preferably from 30 to 70% by weight, based on the total weight of the partially hydrogenated compounds, of compounds of formula (f1) or (f5),
from 20 to 80% by weight, preferably from 30 to 70% by weight, based on the total weight of the partially hydrogenated compounds, of compounds of formula (f2), and
from 20 to 60% by weight, preferably from 30 to 50% by weight, based on the total weight of the partially hydrogenated compounds, of compounds of formula (f7) and/or (f8),

based on the total weight of the partially hydrogenated compounds.

According to an embodiment of the invention, the process is performed at a temperature ranging from 10 to 120° C., preferably from 20° C. to 110° C., more preferably from 30° C. to 100° C., even more preferably from 40° C. to 80° C.

In particular, when the catalyst is a heterogeneous catalyst, the process is preferably performed at a temperature ranging from 40° C. to 80° C.

According to an embodiment of the invention, the process is performed at a hydrogen pressure ranging from 2 bars (2×10⁵ Pa) to 12 bars (12×10⁵ Pa), preferably from 3 bars (3×10⁵ Pa) to 10 bars (10×10⁵ Pa).

When the pressure is of 3 bars or less than 3 bars, the hydrogenation process may be performed in a glass reactor. When the pressure is higher than 3 bars, the hydrogenation process is preferably performed in an autoclave.

Hydrogen can be obtained from any source well known by the skilled person. For example, hydrogen can come from reforming of natural gas, gasification of coal and/or biomass, water electrolysis. After production, hydrogen may be purified via a purification step, for example by pressure swing adsorption.

According to an embodiment, the molar ratio between the conjugated diene compounds and the catalyst is from 500 to 50000, preferably from 1000 to 25000, more preferably from 2000 to 10000.

According to an embodiment of the invention, the process is performed in a solvent, such as methanol or toluene, preferably in toluene. According to an embodiment, the solvent comprises toluene with traces of methanol, i.e. the toluene solvent may comprise less than 1% vol of methanol.

Preferably, the amount of solvent is from 10 to 50 mL for an amount of 5 to 40 mmol of conjugated diene compounds. For example, the amount of solvent if about 30 mL for 10 mmol of conjugated diene compounds.

The reaction mixture may then be analyzed according to any methods known by the skilled person, such as by gas chromatography. An analysis by gas chromatography may allow determining the amount of each isomer of the partially hydrogenated compounds present in the reaction mixture.

### Reaction Mixture

The present invention is also directed to a reaction mixture obtainable by the process of the invention, said reaction mixture comprising:
from 10 to 80% by weight, preferably from 30 to 70% by weight, based on the total weight of the reaction mixture, of compound(s) A resulting from the mono-hydrogenation of compounds of formula (g),
from 20 to 90% by weight, preferably from 30 to 70% by weight, based on the total weight of the reaction mixture, of compound(s) B resulting from the di-hydrogenation of compounds of formula (g),
based on the total weight of the compounds resulting from the partial hydrogenation of compounds of formula (g),

wherein

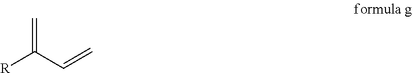

R is a hydrocarbyl radical having 1 to 40 carbon atoms and comprising at least one carbon-carbon double bond, optionally comprising one or more heteroatoms, such as nitrogen, oxygen or sulphur,
   at least 50% by weight, preferably at least 60% by weight, more preferably at least 70% by weight, even more preferably at least 80% by weight, based on the total weight of the compounds A is represented by a compound of formula (g1) or a compound of formula (g5)

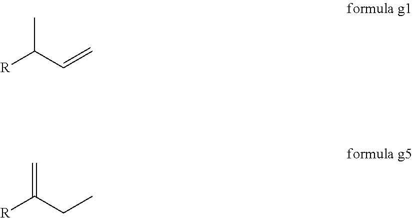

at least 70% by weight, preferably at least 80% by weight, more preferably at least 90% by weight, based on the total weight of the compounds B is represented by a compound of formula (g2):

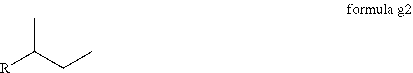

in formulas (g1), (g2) and (g5), R has the same meaning as in formula (g).

Preferably, in the above-formulas, R is a hydrocarbyl radical having from 5 to 20 carbon atoms and comprising at least one carbon-carbon double bond, optionally comprising one or more heteroatoms, such as nitrogen, oxygen or sulphur.

According to a specific embodiment, R consists in carbon and hydrogen atoms.

In one reaction mixture according to the present invention, the R group in each formula (g), (g1), (g2) and (g5) is identical.

Iridium-NHC based catalyst (supported or not supported) was found to allow the recovery of various compositions of olefins (reaction mixtures). In particular, the choice of the iridium metal and the NHC ligand allows the obtaining of a specific reaction mixture. Additionally, the choice of the reaction conditions (temperature, pressure, solvent) allows refining the specific composition of the reaction mixture.

According to an embodiment, in the above-formula (g), R is a hydrocarbyl radical having 1 to 40 carbon atoms and comprising at least two carbon-carbon double bond, optionally comprising one or more heteroatoms, such as nitrogen, oxygen or sulphur. According to this embodiment, the reaction mixture of the invention comprises mono-hydrogenated compounds, di-hydrogenated compounds and tri-hydrogenated compounds.

In particular, according to this embodiment (embodiment wherein the starting conjugated diene compounds comprise at least four carbon-carbon double bonds), the reaction mixture comprises:
from 5 to 70% by weight, preferably from 20 to 70% by weight, more preferably from 30 to 60% by weight, of compound(s) A resulting from the mono-hydrogenation of compounds of formula (g),
from 20 to 80%, preferably from 30 to 70% by weight, more preferably from 40 to 60% by weight, by weight of compound(s) B resulting from the di-hydrogenation of compounds of formula (g), and
from 10 to 70% by weight, preferably from 20 to 60% by weight, more preferably from 30 to 50% by weight, of compound(s) C resulting from the tri-hydrogenation of compounds of formula (g),

based on the total weight of the partially hydrogenated compounds.

According to an embodiment, the reaction mixture of the invention is obtainable by the process of the invention wherein the iridium-NHC based catalyst is in the form of a cationic complex, preferably wherein the iridium-NHC based catalyst responds to formula (I), more preferably formula (Ibis) as defined above. According to this embodiment, the reaction mixture comprises:
from 5 to 70% by weight, preferably from 20 to 70% by weight, more preferably from 30 to 60% by weight, of compound(s) A resulting from the mono-hydrogenation of compounds of formula (g),
from 20 to 80%, preferably from 30 to 70% by weight, more preferably from 40 to 60% by weight, by weight of compound(s) b resulting from the di-hydrogenation of compounds of formula (g), and
from 10 to 70% by weight, preferably from 20 to 60% by weight, more preferably from 30 to 50% by weight, of compound(s) C resulting from the tri-hydrogenation of compounds of formula (g),

based on the total weight of the partially hydrogenated compounds,

wherein at least 50% by weight, preferably at least 60% by weight, more preferably at least 70% by weight, even more preferably at least 80% by weight, based on the total weight of the compounds A is represented by a compound of formula (g1).

According to an embodiment, the reaction mixture further comprise as compound(s) A resulting from the mono-hydrogenation of compounds of formula (g), compounds of formula (g3) and/or of formula (g4):

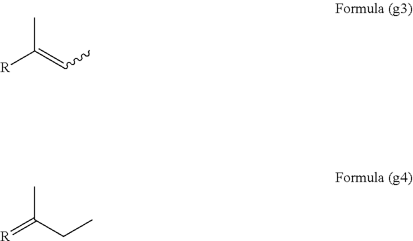

In formula (g3), R is a hydrocarbyl radical having 1 to 40 carbon atoms and comprising at least one carbon-carbon double bond, optionally comprising one or more heteroatoms, such as nitrogen, oxygen or sulphur.

In formula (g4), R′ represents the group R with one hydrogen atom in less (since R′ is linked to the previously conjugated diene function with a carbon-carbon double bond).

According to a preferred embodiment, the compounds of formula (g) are selected from terpenes.

Preferably, the terpenes are selected from farnesene. Preferably, the reaction mixture of the invention comprises:
from 5 to 70% by weight, preferably from 10 to 60% by weight, more preferably from 20 to 50% by weight, of mono-hydrogenated farnesene,
from 20 to 80% by weight, preferably from 30 to 70% by weight, more preferably from 40 to 60% by weight, of di-hydrogenated farnesene,
from 10 to 70% by weight, preferably from 20 to 60% by weight, more preferably from 30 to 50% by weight, of tri-hydrogenated farnesene,

based on the total weight of the reaction mixture,

wherein at least 50% by weight, preferably at least 60% by weight, more preferably at least 70% by weight, even more preferably at least 80% by weight, based on the total weight of the mono-hydrogenated farnesene is represented by a compound of formula (f1) or (f5),

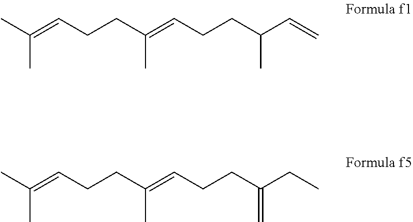

and wherein at least 70% by weight, preferably at least 80% by weight, more preferably at least 90% by weight, based on the total weight of the di-hydrogenated farnesene is represented by a compound of formula (f2),

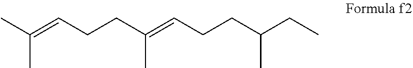

Preferably, at least 60% by weight, more preferably at least 70% by weight, even more preferably at least 80% by weight, ideally at least 90% by weight, based on the total weight of the di-hydrogenated farnesene, is represented by a compound of formula (f2).

Preferably, at least 50% by weight, preferably at least 60% by weight, more preferably at least 70% by weight, even more preferably at least 80% by weight, ideally at least 90% by weight, based on the total weight of the tri-hydrogenated farnesene, is represented by compounds of formula (f7) and/or (f8),

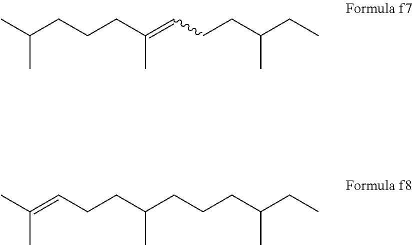

According to an embodiment, the reaction mixture of the invention is obtainable by the process of the invention wherein the iridium-NHC based catalyst is in the form of a cationic complex, preferably wherein the iridium-NHC based catalyst responds to formula (I), more preferably formula (Ibis) as defined above. According to this embodiment, the reaction mixture comprises:
from 5 to 70% by weight, preferably from 10 to 60% by weight, more preferably from 20 to 50% by weight, of mono-hydrogenated farnesene,
from 20 to 80% by weight, preferably from 30 to 70% by weight, more preferably from 40 to 60% by weight, of di-hydrogenated farnesene,
from 10 to 70% by weight, preferably from 20 to 60% by weight, more preferably from 30 to 50% by weight, of tri-hydrogenated farnesene,

based on the total weight of the partially hydrogenated farnesene,

wherein at least 50% by weight, preferably at least 60% by weight, more preferably at least 70% by weight, even more preferably at least 80% by weight, based on the total weight of the mono-hydrogenated farnesene is represented by a compound of formula (f1).

In particular, the reaction mixture of the invention may comprise:
from 5 to 70% by weight, preferably from 10 to 60% by weight, more preferably from 20 to 50% by weight, of mono-hydrogenated farnesene,
from 20 to 80% by weight, preferably from 30 to 70% by weight, more preferably from 40 to 60% by weight, of di-hydrogenated farnesene,
from 10 to 70% by weight, preferably from 20 to 60% by weight, more preferably from 30 to 50% by weight, of tri-hydrogenated farnesene,

based on the total weight of the partially hydrogenated farnesene,

wherein the compounds of formula (f1) represent from 50% to 90% by weight of the mono-hydrogenated farnesene, and the compounds of formula (f3) represent from 10 to 40% by weight based on the total weight of the mono-hydrogenated farnesene.

The products contained in the reaction mixture may be further separated and/or purified by any methods known by the one skill in the art.

The reaction mixture of the invention and/or the separated/purified products resulting therefrom, may be used for the preparation of plastics, detergents, lubricants, or oils. In particular, the reaction mixture of the invention may be polymerized, oligomerized, copolymerized or co-oligomerized to make for example an oil, a lubricant or a resin. They may also be functionalized in order to make them suitable for specific applications.

The reaction mixture according to the invention and/or derivatives thereof may be used in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as EDM (Electronic Discharge Machining) fluids, rust preventive in industrial lubricants, as extender oils, as drilling fluids, as industrial solvents, as viscosity depressants in plasticized polyvinyl chloride formulations, as crop protection fluids.

### EXAMPLES

### Example 1

### Preparation of the Supported Catalyst of Formula (III), in Particular of Formula (IIIbis)

The supported mono-NHC-Iridium complexes are obtained starting from the imidazolium functionnalized material. This latter is obtained by cocondensation of tetraethylorthosilicate (TEOS) and iodopropyltriethoxysilane (IC₃H₆Si(OEt)₃) in a hydrolytic sol-gel process in the presence of Pluronic 123 as structure-directing agent. This material is then treated with mesitylimidazole to generate the corresponding imidazolium functionalities and then with Me₃SiBr/NEt₃ to transform the surface silanol groups into trimethylsiloxane moieties. Thus, the Imidazolium containing material is treated with AgOC(CF₃)₃ to give the silver-NHC supported complex. The corresponding supported Iridium-mono-NHC complex is obtained upon transmetallation with the iridium precursor.
The general scheme of the imidazolium containing material and the NHC—Ag supported complex may be depicted below:

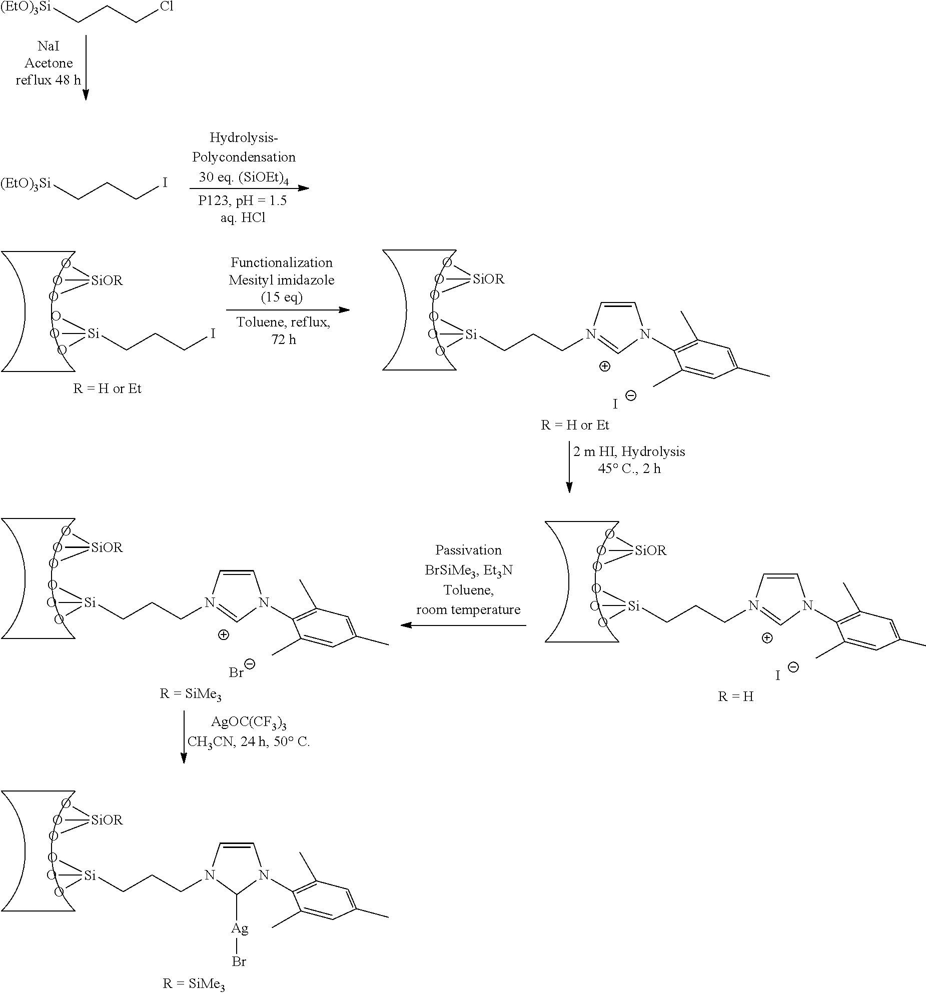

Supported Iridium-complexes may then be synthesized from the material-NHC—Ag—X supported complex following the synthetic pathways represented below:

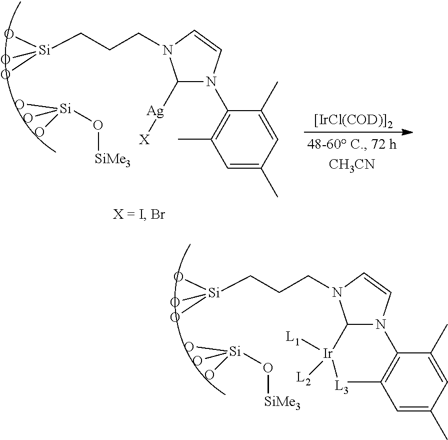

### Example 2

### Hydrogenation Process

The process according to the present invention has been performed using beta-farnesene as conjugated diene compounds.

Depending on the pressure used, two protocols have been performed:

Typical Procedure for Catalytic Tests in Glass Reactor (3 Bars H₂ and Less Than 3 Bars):

All experiments were performed following the same procedure in a specifically adapted 200 mL glass reactor with connection to vacuum, argon and dihydrogen lines. The homogeneous catalyst (0.01 mmol), β-Farnesene (10 mmol, 2.4 g) and dodecane (5 mmol, 0.85 g) used as internal standard, were introduced into the reactor. The degassed solvent (toluene 30 mL) is then added thanks to a syringe into the reactor. The closed reactor was first purged with argon and then with the H₂ gas mixture. The reaction mixture was placed at the desired temperature and then under 3 bar H₂ with a 600 rpm stirring rate. Samples were taken during the experiment in order to follow the reaction course by gas chromatography (the reactor is slowly depressurized, put under argon atmosphere, and then pressurized at 3 bar H₂). At the end of the reaction, the reactor was cooled to room temperature and then slowly depressurized. The crude mixture was analyzed by gas chromatography.

Typical Procedure for Catalytic Tests in Autoclave (10 Bars H2 and More Than 10 Bars):

All experiments were performed following the same procedure in a 90 mL stainless-steel autoclave. The supported catalyst (10mg) was suspended in β-Farnesene (10 mmol, 2.04 g) and dodecane (5 mmol, 0.85g) used as internal standard. The mixture was introduced in the reactor, followed by the solvent (Toluene, 30 mL). The closed reactor was then purged three times with the H2 gas mixture. The reaction mixture was placed under 10 bar H2 and heated until the desired temperature with a 800 rpm stirring rate. The pressure was then completed until 30 bar H2. The experiment was running under a continuous feed of gas mixture. Samples were taken during the experiment in order to follow the reaction course by gas chromatography. At the end of the reaction, the autoclave was cooled to room temperature and then slowly depressurized. The crude mixture was analyzed by gas chromatography.

### Ex. 2a

### Hydrogenation Process Using Homogeneous Catalysts and a Heterogeneous Catalyst (M—Ir—NHC)

Three different cationic iridium complexes of the general formula [Ir (COD)(NHC)(phosphane)]X have been tested. Each cationic iridium complex responds to the developed formula:

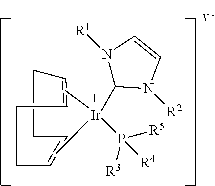

wherein R¹ and R² are mesityl groups (Mes),

X represents a hexafluorophosphate,

R³, R⁴ and R⁵ represents ligands selected from methyl, phenyl or Benzyl groups.

One neutral iridium-NHC complex has also been tested: [IrCl(COD)(MesPrIm)], where Pr represents a propyl group.

The iridium complexes have been evaluated in homogeneous catalysis at different reaction conditions: temperature (18° C. or 30° or 50° C.), H₂ pressure (10 bar or 3 bar), and solvent (methanol or toluene).

Typical conditions: Minimum molar ratio farnesene/iridium=1000; 30 mL solvent; 10 mmol Farnesene. Reaction performed in a Fisher-Porter tube at desired H₂ pressure and temperature.

Examples of selectivity obtained are given in the table below. The global selectivity refers to the weight percentage of the mono-hydrogenated compounds (206) and to the weight percentage of the di-hydrogenated compounds (208) based on the total weight of the partially hydrogenated compounds in the reaction mixture.

The selectivity/206 refers to the weight percentage of each mono-hydrogenated compound with respect to the weight of all the mono-hydrogenated compounds.

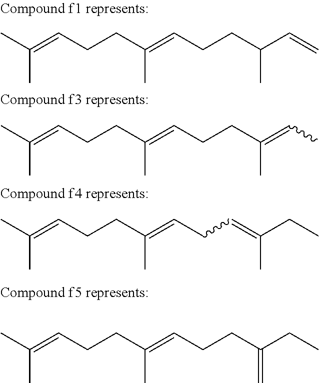

The farnesene conversion refers to the amount in percentage by weight of farnesene that have reacted.

TABLE 1
Conversion and selectivities obtained with the process of the invention

| | | Global | | | | | |
|---|---|---|---|---|---|---|---|
| | time | selectivity | | Selectivity/206 | | | |
| | (farnesene | (%) | | (%) | | | |
| Iridium complex/conditions | conversion %) | 206 | 208 | f1 | f3 | f4 | f5 |
| [Ir(COD)(MesMesIm)PBn₃]PF₆ | 1 h 30 | 40.7 | 58.5 | 78.9 | 11.3 | 2.5 | 7.3 |
| CAS No. 1019853-01-0 | (~61%) | | | | | | |
| MeOH | 30 min | 44.2 | 55.8 | 77.1 | 10.7 | 7.0 | 5.1 |
| 18° C.-3 bar | (~25%) | | | | | | |
| [Ir(COD)(MesMesIm)PPhMe₂]PF₆ | 1 h 30 | 32.1 | 63.7 | 71.3 | 18.0 | 6.2 | 4.4 |
| CAS No. 1019853-03-2 | (~64%) | | | | | | |
| Toluene | 30 min | 32.4 | 61.0 | 81.7 | 18.3 | 0.0 | 0.0 |
| 50° C.-3 bar | (~7%) | | | | | | |
| [Ir(COD)(MesMesIm)PPh₃]PF₆ | 20 min | 39.5 | 60.5 | 77.6 | 15.6 | 6.8 | 0.0 |
| CAS No. 1019852-99-3 | (~18%) | | | | | | |
| Toluene | 1 h 40 | 30.5 | 69.5 | 65.8 | 21.3 | 8.8 | 4.1 |
| 30° C.-3 bar | (~82%) | | | | | | |
| [IrCl(COD(MesPrIm)] | 3 h 15 | 45.7 | 37.5 | 23.2 | 4.1 | 2.6 | 70.1 |
| Toluene | (~48%) | | | | | | |
| 50° C.-3 bar | | | | | | | |

With the cationic iridium complexes, the mono-hydrogenated product f1 is present in majority among mono-hydrogenated products (molecular mass 206). Said selectivity is present all along the reaction process, i.e. at the beginning of the reaction but also after 1 h30 (at 100% farnesene conversion).

In the table 2 below, the same tests have been performed and continued in order to obtain about 100% of farnesene conversion.

Additionally, another Ir—NHC based catalyst has been tested. This M—Ir—NHC based catalyst is a neutral iridium catalyst supported on silica, responding to formula (IIIbis), wherein the ligands are Cl and NHC (with R¹=propylene linker and R²=mesityl group). This catalyst is similar to the catalyst used in example 2b below.

In table 2, below, “206” refers to the mono-hydrogenated farnesene, “208” refers to the di-hydrogenated farnesene, “210” refers to the tri-hydrogenated farnesene and 212 refers to the saturated farnesane.

TABLE 2
Reaction mixtures

| | | | Global selectivity per weight | | | |
|---|---|---|---|---|---|---|
| | Time - | | (%) | | | |
| Catalyst/conditions | (conversion) | | 206 | 208 | 210 | 212 |
| [Ir(COD)(MesMesIm)PBn₃]PF₆ | 1 h 30 | (~61%) | 40.7 | 58.5 | 0.0 | 0.0 |
| MeOH | | | | | | |
| 18° C. | 3 h 15 | (~100%) | 8.0 | 91.0 | 0.6 | 0.0 |
| 3 bar | | | | | | |
| [Ir(COD)(MesMesIm)PPhMe₂]PF₆ | 1 h 30 | (~64%) | 32.1 | 63.7 | 2.2 | 1.0 |
| Toluene | 3 h 15 | (100%) | 3.4 | 30.2 | 53.4 | 11.5 |
| 50° C.-3 bar | | | | | | |
| [IrClCOD(MesPrIm)] | 3 h 15 | (48%) | 45.7 | 37.5 | 12.9 | 0.5 |
| Toluene | | | | | | |
| 50° C.-3bar | | | | | | |
| M-Ir-NHC | 1 h | (~100%) | 30.1 | 53.3 | 14.4 | 2.17 |
| (Ir-NHC supported on silica) | | | | | | |
| Toluene | | | | | | |
| 70° C. | | | | | | |
| 10 bar | | | | | | |

For catalysts in the form of a cationic complex, we note that the compound of formula (f1) is mainly obtained, followed by the compound of formula (f3), among the total amount of the mono-hydrogenated (206 products) obtained.

For catalyst in the form of a neutral complex, we note that the compound of formula (f5) is mainly obtained, followed by the compound of formula (f1), among the total amount of the mono-hydrogenated (206 products—obtained.

We observe that after 3 h15 of reaction, there is a high selectivity towards the di-hydrogenated compounds 208. The high selectivity towards di-hydrogenated compounds is even more observed when the process is performed in methanol.

For catalyst in the form of a cationic complex, we also observe that at 100% of farnesene conversion, the main compounds are the tri-hydrogenated compounds when the process is performed in toluene.

### Ex. 2b

### Hydrogenation Process Using a Heterogeneous Catalyst

Further tests have been performed to evaluate the activity of the supported catalyst of formula (IIIbis):

Supported neutral iridium catalyst M-Ir:
Support=silica,
Ligands: Cl and NHC (with R¹=propylene linker and R²=mesityl group)

The catalyst of formula (IIIbis) tested in the present example has been prepared according to a method such as described above.

The neutral iridium supported catalyst has been evaluated in the following conditions:

Pressure H₂: 3 bar;

3 bar H₂ in Fisher Porter tube or glass reactor 10 bar in autoclave;

Temperature 30° C.-50° C-70° C.;

Toluene or MeOH as solvent;

Molar Ratio farnesene/Iridium: minimum 3000.

In the following examples, the catalyst used responds to the following formula:

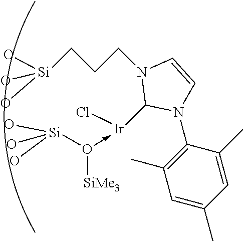

In the above-formula, the surface plays the role of ligands (as represented by the arrow in the formula).
Influence of H₂ pressure: an increase of the pressure from 3 to 10 bars at 50° C. allows to significantly increase the activity; after 1 h at 10 bar the farnesene conversion is above 90% whereas said conversion is below 10% at 3 bar.
Supported complex is more active in hydrogenation than homogeneous complex: initial Trun Over Frequency TOF (at 15 min) is multiplied by two with a supported complex.
Influence of temperature: at 50° C. under 10 bar H_{2,} in 1 h the maximal Turn Over Number TON (=mol of converted farnesene/mol of catalyst) is almost reached (3000 for supported complex and 1000 for homogeneous complex). TOF=TON/time.
As illustrated in table 3 below, selectivities can be modulated by changing the temperature:
   at 30° C. (for the supported complex), under 10 bar H_{2,} the hydrogenation is less important (conversion of 24.6%) and the molecule (f2) is present at almost 60% by weigh in the reaction mixture.
   at 50° C., 10 bar H_{2,} after 3h, a mixture of products is obtained: farnesane, 2 products with molecular mass 210 (=tri-hydrogenated products), 2 products with molecular mass 208 (one of them is the molecule f2) and 3 products with molecular mass 206 (molecules (f1), (f5) and (f3); molecules (f1) being the major 206 product). The major product (35%) among the partially hydrogenated compounds is the molecule (f2).

In table 3 below, the detection of each compound has been performed using a gas chromatographic column DB23 (from Agilent) with the following temperature program:

initially the temperature is of 80° C. during 5 min,

then the temperature is increased of 15° C/min until 120° C.,

then the temperature is increased of 3° C/min until 220° C.

In the table 3 below, the “Rt” line refers to the retention time of each compounds expressed in minute.

TABLE 3
selectivities with a supported catalyst

| | | Global selectivity per weight (%) | | | | | | | | |
|---|---|---|---|---|---|---|---|---|---|---|
| | | 212 | 210 | 210 | 208 | | 206 | | | |
| Catalyst/ | Time (% | Rt | | | | | | | | |
| conditions | conversion) | 11.537 | 12.60 | 13.07 | 14.16 f2 | 14.72 | 14.29 f1 | 15.45 f3 | 15.59 f4 | 15.82 f5 |
| M-Iridium | 5 h (24.6 %) | 1.79 | 3.03 | 9.26 | 58.20 | 1.15 | 3.82 | 4.98 | 1.81 | 9.08 |
| 30° C. | | | | | | | | | | |
| 10 bar H₂ | | | | | | | | | | |
| Toluene | | | | | | | | | | |
| M-Iridium | 4 h (100 %) | 13.12 | 20.43 | 17.08 | 36.99 | 2.58 | 0.32 | 0.40 | 1.24 | 6.97 |
| 50° C. | | | | | | | | | | |
| 10 bar H₂ | | | | | | | | | | |
| Toluene | | | | | | | | | | |

We also observe that when the temperature of the process is less than or equal to 30° C., the process leads to a reaction mixture that comprises at least 50% by weight of di-hydrogenated compounds, in particular at least 60% by weight of di-hydrogenated compounds, based on the total weight of the partially hydrogenated compounds.

In particular, when the temperature of the process is less than or equal to 30° C., the process leads to a reaction mixture that comprises at least 50% by weight of di-hydrogenated compounds of formula (g2), in particular at least 60% by weight of di-hydrogenated compounds of formula (g2), based on the total weight of the partially hydrogenated compounds.

1. A process for partial hydrogenation of conjugated diene compounds comprising at least one conjugated diene function and at least one additional carbon-carbon double bond, the process comprising reacting the conjugated diene compounds with hydrogen in the presence of an Iridium-NHC based catalyst, to produce a reaction mixture comprising partially hydrogenated compounds.

2. The process according to claim 1, wherein the at least one conjugated diene function is a terminal conjugated diene function.

3. The process according to claim 1, wherein a portion of the partially hydrogenated compounds results from mono-hydrogenation of one carbon-carbon double bond of the conjugated diene function.

4. The process according to claim 1, wherein a portion of the partially hydrogenated compounds results from di-hydrogenation of two carbon-carbon double bonds of the conjugated diene function.

5. The process according to claim 1, wherein the conjugated diene compounds comprising at least one conjugated diene function and at least one additional carbon-carbon double bond are selected from terpenes.

6. The process according to claim 1, wherein the hydrogenation is performed at a temperature ranging from 10° C. to 120 ° C.

7. The process according to claim 1, wherein the hydrogenation is performed at a pressure ranging from 2 bars to 12 bars.

8. The process according to claim 1, wherein the reaction mixture comprises mono-hydrogenated compounds wherein a mono-hydrogenated compound resulting from the hydrogenation of one carbon-carbon double bond of the conjugated diene function represents at least 50% by weight of the total weight of the mono-hydrogenated compounds.

9. The process according to claim 1, wherein the conjugated diene compound is a compound of formula (g): 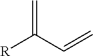
wherein, R is a hydrocarbyl radical having 1 to 40 carbon atoms and comprising at least one carbon-carbon double bond.

10. The process according to claim 9, wherein the partially hydrogenated compounds comprise:
the compound of formula (g1) or (g5) represents at least 50% by weight of the total weight of the mono-hydrogenated compounds, wherein 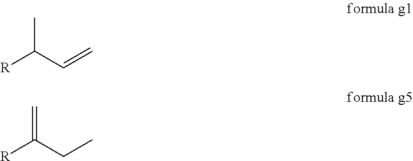
the compound of formula (g2) represents at least 50% by weight of the total weight of the di-hydrogenated compounds, wherein 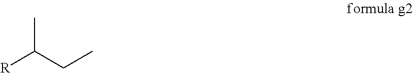
in formulas (g1), (g5) and (g2), R represents the same group as in formula (g).

11. The process according to claim 1, wherein the iridium-NHC based catalyst is a homogeneous catalyst.

12. The process according to claim 11, wherein the homogeneous catalyst is a complex of formula (I) or of formula (II):
[Ir(L1)(L2)(NHC)(L3)]X (I)
[Ir(L′1)(L′2)(NHC)(L′3)] (II)
wherein
L1, L2 and L3 are independently to each other a ligand,
L′1, L′2 and L′3 are independently to each other a ligand, and
X is a non-coordinated counter-anion.

13. The process according to claim 1, wherein the iridium-NHC based catalyst is a heterogeneous catalyst.

14. The process according to claim 13, wherein the heterogeneous catalyst is a silica-supported iridium-NHC based catalyst of formula (Ill): 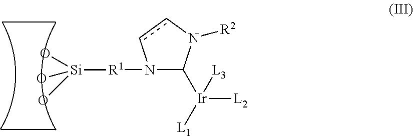
wherein:
L₁, L₂ and L₃ are independently to each other a ligand,
R¹ represent an alkylene or an arylene group optionally substituted, and
R² represents an alkyl or an aryl group optionally substituted.

15. The process according to claim 12, wherein the catalyst is a complex of formula (I) and wherein the reaction mixture is such that the compound of formula (g1) represents at least 50% by weight of the total weight of the mono-hydrogenated compounds.

16. A reaction mixture comprising:
from 20 to 80% by weight of compound(s) A resulting from a mono-hydrogenation of compounds of formula (g),
from 20 to 80% by weight of compound(s) B resulting from a di-hydrogenation of compounds of formula (g),
based on the total weight of the partially hydrogenated compounds,
wherein 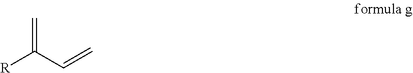
R is a hydrocarbyl radical having 1 to 40 carbon atoms and comprising at least one carbon-carbon double bond,
at least 50% by weight based on the total weight of the compounds A is represented by a compound of formula (g1) or a compound of formula (g5) 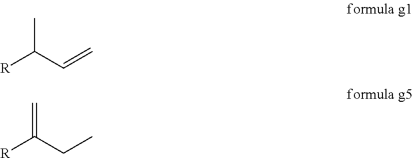
at least 70% by weight based on the total weight of the compounds B is represented by a compound of formula (g2) 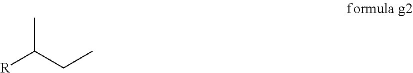
in formulas (g1), (g2) and (g5), R has the same meaning as in formula (g).

17. The reaction mixture according to claim 16, wherein the R group of the compounds of formula (g) is a hydrocarbyl radical having 1 to 40 carbon atoms and comprising at least two carbon-carbon double bonds,
and wherein the reaction mixture comprises:
from 5 to 70% by weight of compound(s) A resulting from the mono-hydrogenation of compounds of formula (g),
from 20 to 80% by weight of compound(s) b resulting from the di-hydrogenation of compounds of formula (g), and
from 10 to 70% by weight of compound(s) C resulting from the tri-hydrogenation of compounds of formula (g),
based on the total weight of the partially hydrogenated compounds.

18. The reaction mixture according to claim 17, wherein the compound of formula (g) is farnesenes, the reaction mixture comprising:
from 5 to 70% by weight of compound(s) A resulting from the mono-hydrogenation of farnesene,
from 20 to 80% by weight of compound(s) B resulting from the di-hydrogenation of farnesene, and
from 10 to 70% by weight of compound(s) C resulting from the tri-hydrogenation of farnesene,
based on the total weight of the partially hydrogenated farnesene,
wherein at least 50% by weight based on the total weight of the mono-hydrogenated farnesene is represented by a compound of formula f1 or f5: 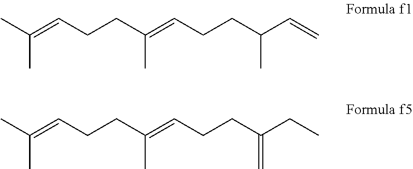
and wherein at least 70% by weight based on the total weight of the di-hydrogenated farnesene is represented by a compound of formula f2:

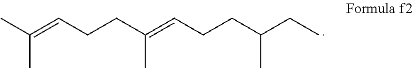

19. A reaction mixture or a derivative thereof, in sealants or polymers formulation with silicone, in coating fluids, in metal extraction, in mining, in explosives, in concrete demoulding formulations, in adhesives, in printing inks, in metal working fluids, in resins, in pharmaceutical products, in paint compositions, in polymers used in water treatment, paper manufacturing or printing pastes and cleaning solvents, as cutting fluids, as rolling oils, as Electronic Discharge Machining (EDM) fluids, rust preventive in industrial lubricants, as extender oils, as drilling fluids, as industrial solvents, as viscosity depressants in plasticized polyvinyl chloride formulations, or as crop protection fluids,
the reaction mixture comprising:
from 20 to 80% by weight of compound(s) A resulting from a mono-hydrogenation of compounds of formula (g),
from 20 to 80% by weight of compound(s) B resulting from a di-hydrogenation of compounds of formula (g),
based on the total weight of the partially hydrogenated compounds,
wherein 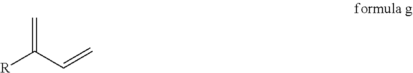
R is a hydrocarbyl radical having 1 to 40 carbon atoms and comprising at least one carbon-carbon double bond,
at least 50% by weight based on the total weight of the compounds A is represented by a compound of formula (g1) or a compound of formula (g5) 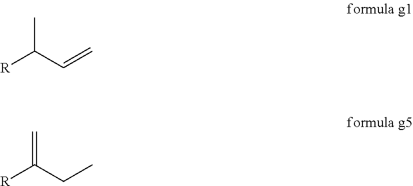
at least 70% by weight based on the total weight of the compounds B is represented by a compound of formula (g2) 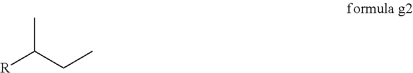
in formulas (g1), (g2) and (g5), R has the same meaning as in formula (g).
